(19) United States
(12) Patent Application Publication
(10) Pub. No.: US 2019/0209356 A1
(43) Pub. Date: Jul. 11, 2019

(54) ENDOLUMINAL DELIVERY DEVICE ASSEMBLY

(71) Applicant: Cook Medical Technologies LLC, Bloomington, IN (US)

(72) Inventors: James Collins, Paddington (AU); Logan Smith, Mount Gravatt (AU)

(21) Appl. No.: 16/244,653

(22) Filed: Jan. 10, 2019

Related U.S. Application Data
(60) Provisional application No. 62/615,720, filed on Jan. 10, 2018.

(30) Foreign Application Priority Data
Jan. 10, 2018 (AU) 2018900064

(51) Int. Cl. A61F 2/966; A61M 25/09

(52) U.S. Cl. CPC A61F 2/966; A61F 2/07; A61M 25/09041

(57) ABSTRACT

An endoluminal delivery device assembly is disclosed. The assembly comprises: an introducer including a seal housing assembly, housing a seal, and an sheath extending proximally therefrom; a delivery device including a handle body and a pusher catheter extending from the body, the pusher catheter having a pusher catheter external surface, the pusher catheter extending through the introducer such that the external surface slideably engages with the seal; a guide wire catheter extending through the body and through the pusher, the guide wire catheter being affixed at a proximal end thereof to a tip assembly; and a linking portion for linking the body and the seal housing assembly together, the linking portion having an unlinked condition and a linked condition. In the linked condition, sliding movement between the pusher catheter and the seal is limited such that disengagement between the pusher catheter external surface and the seal is prevented.

### CROSS REFERENCE TO RELATED APPLICATIONS

This application claims the benefit of Australian patent application No. 2018900064 filed on Jan. 10, 2018 entitled, “An Endoluminal Delivery Device Assembly” and U.S. provisional application No. 62/615,720 filed on Jan. 10, 2018 entitled, “An Endoluminal Delivery Device Assembly” the entire contents of which are incorporated herein by reference.

### TECHNICAL FIELD

The present invention relates to endografts and their delivery systems, sometimes referred to as endoluminal delivery device assemblies. In particular, the present invention relates to endoluminal delivery device assemblies capable of delivering prostheses, endografts or stent grafts into the vascular system of humans or animals.

### BACKGROUND OF THE INVENTION

Stent graft and delivery devices are used in aortic intervention. They are used by vascular surgeons to treat aneurysms and to repair regions of the aorta, including the aortic arch, the thoracic aorta, the abdominal aorta and the aortic bifurcation.

Delivery devices allow deployment of intraluminal prostheses or endografts into the lumen of a patient from a remote location.

Numerous devises and procedures have been developed that involve the percutaneous insertion of a prosthesis into a body lumen, such as a blood vessel or duct, of a patient's body. Such a prosthesis may be introduced into the lumen by a variety of known techniques. For example, a wire guide may be introduced into a blood vessel using the Seldinger technique. This technique involves creating a surgical opening in the vessel with a needle and inserting a wire guide into the vessel through a bore of the needle. The needle can be withdrawn, leaving the wire guide in place. A delivery device is then inserted over the wire guide and into the vessel. The delivery device may be used in conventional fashion to insert into the blood vessel a variety of prostheses, such as stents, stent grafts, catheters, cardiac leads, balloons, and the like.

For example, the delivery device may be used to deliver and deploy an expandable prosthesis, such as a stent graft, to an aneurysmal blood vessel site. A stent graft is usually formed from a tubular body of a biocompatible graft material with one or more stents mounted into or onto the tubular body to provide support therefor. The stents may be balloon expandable stents and/or self-expanding stents. The deployment of the prosthesis into the lumen of a patient from a remote location by the use of an introducer delivery and deployment device is described in, e.g., U.S. Pat. No. 7,435,253 to Hartley entitled “A Prosthesis and a Method and Means of Deploying a Prosthesis,” which is incorporated herein by reference in its entirety.

Delivery devices are configured to retain a prosthesis in a delivery configuration during delivery to the desired deployment site. The delivery catheter typically includes an inner catheter/cannula spaced from an outer sheath to define a prosthesis retaining region for receiving the prosthesis. The prosthesis is loaded onto an inner cannula along a prosthesis retaining region, with an outer sheath retaining the prosthesis in the delivery configuration. After the delivery device is delivered to the desired deployment site, the prosthesis may be deployed, for example, with retraction of the outer sheath relative to the inner cannula away from the prosthesis to allow for expansion thereof. Accurate placement of an appropriately sized prosthesis generally sufficiently covers the target site for treatment and the ends of the prosthesis are typically engaged with healthy tissue of the body lumen.

Endovascular delivery devices require significant expertise and experience to operate. Correct sequencing of various manual operations performed outside the body (at a distal end of a delivery device) are required for successful and optimum deployment of an endograft. It is desirable to make operation as intuitive and foolproof as possible.

For endovascular delivery devices that include a pusher catheter and a pusher catheter seal, uncontrolled or inadvertent withdrawal of the pusher catheter through the pusher catheter seal can lead to sealing problems and increase the likelihood or severity of blood loss where the devices are used within arteries.

It is an object of the invention to provide an improved endograft and delivery device assembly.

Throughout this specification, the term “distal” with respect to a portion of the aorta, a deployment device or an endograft means the end of the aorta, deployment device or endograft further away in the direction of blood flow from the heart and the term “proximal” means the portion of the aorta deployment device or end of the endograft nearer to the heart in the direction of blood flow.

### SUMMARY OF THE INVENTION

According to an aspect of the invention, an endoluminal delivery device assembly comprises:
an introducer, the introducer including a seal housing assembly and an elongate sheath extending proximally from the seal housing assembly, the seal housing assembly housing a pusher catheter seal;
a delivery device, the delivery device including a handle body and an elongate pusher catheter extending proximally from the handle body, the pusher catheter having a pusher catheter external surface, the pusher catheter extending through the introducer such that the pusher catheter external surface slideably engages with the pusher catheter seal;
a tip assembly at a proximal end of the delivery device;
a guide wire catheter extending through the handle body and through the pusher, the guide wire catheter being affixed at a proximal end thereof to the tip assembly;
an endograft receiving portion for receiving the endograft between the tip assembly and the pusher; and
a linking portion for linking the handle body and the seal housing assembly together, the linking portion mounted between the handle body and the seal housing assembly, the linking portion having an unlinked condition and a linked condition,
whereby, in the linked condition, sliding movement between the pusher catheter and the pusher catheter seal is limited such that disengagement between the pusher catheter external surface and the pusher catheter seal is prevented.

In one form, the linking portion is slideably mounted to the handle body.

In one form, when in the linked condition, the linking portion is joined to the seal housing assembly.

In one form the assembly further comprises a joiner assembly for joining the linking portion to the seal housing assembly.

In one form the joiner assembly comprises:
a first mating joiner feature on the linking portion; and
a second mating joiner feature on seal housing assembly.

In one form the pusher catheter includes a proximal end, and wherein the assembly comprises:
an first condition in which the proximal end of the pusher catheter is spaced from the tip assembly at a first spacing for accommodating a compressed endograft; and
a second condition in which the proximal end of the pusher catheter is spaced from the tip assembly at a second spacing, the second spacing shorter than the first spacing.

In one form the sheath includes a proximal end, and wherein the assembly has a third condition in which the proximal end of the sheath is adjacent to the tip assembly.

In one form, when in the linked condition, the limited sliding movement between the pusher catheter and the pusher catheter seal is sufficient to bring the proximal end of the sheath into engagement with the tip assembly.

In one form the assembly further comprises a lock, the lock comprising:
a locking actuator having a locking projection; and
a locking recess,
wherein the lock has a locked condition in which the locking projection engages the locking recess thereby locking the linking portion to the handle body so as to prevent relative sliding movement.

In one form the actuator is moveable to an unlocked condition in which the locking projection is disengaged from the locking recess.

In an alternative form, when in the unlinked condition, the linking portion is slideable mounted to the seal housing assembly.

In one form, the linking portion is slideably mounted to the seal housing assembly.

In one form, when in the linked condition, the linking portion is joined to the handle body.

In one form the assembly further comprises a joiner assembly for joining the linking portion to the handle body.

In one form the joiner assembly comprises:
a first mating joiner feature on the linking portion; and
a second mating joiner feature on handle body.

### BRIEF DESCRIPTION OF THE DRAWINGS

Embodiments of the present invention will be discussed with reference to the accompanying drawings wherein:

FIG. 1A shows a first embodiment of an endoluminal delivery device assembly according to the invention in a side view;

FIG. 1B shows a proximal end of the assembly of FIG. 1A;

FIG. 2A is a similar side view to that of FIG. 1A but showing a sheath assembly in a retracted position;

FIG. 2B shows a proximal end of the assembly of FIG. 2A;

FIG. 3A is a similar side view to that of FIG. 2A but showing an endograft in a partially released condition;

FIG. 3B shows a proximal end of the assembly of FIG. 3A;

FIG. 4A is a similar side view to that of FIG. 3A but showing an endograft in a fully released condition;

FIG. 4B shows a proximal end of the assembly of FIG. 4A;

FIG. 5A is a similar side view to that of FIG. 4A but showing a tip assembly of the delivery assembly retracted back into engagement with a proximal end of a sheath;

FIG. 5B shows a proximal end of the assembly of FIG. 5A;

FIG. 6 is a close-up isometric view showing the delivery assembly of FIG. 1A;

FIG. 7 is a similar view to that of FIG. 6, but shows the delivery assembly after its sheath assembly has been withdrawn into the position shown in FIG. 2A;

FIG. 8 is a close-up isometric view similar to that of FIG. 7;

FIG. 9 is a detailed cross-sectional view showing the delivery assembly just before engagement of the sheath assembly with the handle assembly;

FIG. 10A is a similar view to that of FIG. 9, but showing the delivery assembly after engagement of the sheath assembly with the handle assembly;

FIG. 10B is a close up view of the circled portion 10B of FIG. 10A;

FIG. 11 is a close-up isometric view of the assembly shown in FIG. 5A in the configuration shown in FIG. 5A;

FIG. 12 is a similar view to FIG. 11, but shows the detailed side view of a portion of the assembly illustrated in FIG. 11;

FIG. 13 shows a portion of the assembly in the position shown in FIG. 12 but in a detailed cross-sectional view;

FIGS. 14A and 14B show close-up isometric views of a proximal end of a handle body that can be seen in FIGS. 11 and 12;

FIGS. 15A and 15B are end and isometric views respectively of a linking portion of the delivery assembly shown in FIGS. 11 and 12 for instance;

FIG. 16 is a detailed isometric view of a component of the handle body shown in FIGS. 14A and 14B;

FIGS. 17A and 17B show components of the proximal end of the handle body shown in FIGS. 14A and 14B;

FIG. 18 is a detailed isometric view showing the inner handle portion of the assembly shown in FIGS. 1A to 5A and FIG. 9;

FIG. 19 is an isometric view of a second embodiment of a delivery assembly according to the invention in a position corresponding to the position of the first embodiment shown in FIG. 1A;

FIG. 20 is a similar view to that of FIG. 19, but shows the delivery assembly after its sheath assembly has been withdrawn into the position corresponding to the position of the first embodiment shown in FIG. 2A;

FIG. 21 is a detailed cross-sectional view showing the delivery assembly just before mating of joiner features;

FIGS. 22A is a similar detailed cross-sectional view to FIG. 21, but showing the delivery assembly just after mating of joiner features;

FIGS. 22B is a close up view of a portion of FIG. 22A;

FIG. 23 shows the assembly in a position corresponding to the position of the first embodiment shown in FIG. 4A in the configuration shown in FIG. 4A; and

FIG. 24 shows the assembly in a position corresponding to the position of the first embodiment shown in FIG. 5A in the configuration shown in FIG. 5A.

### DETAILED DESCRIPTION

For the purposes of understanding the principles of the invention, reference will now be made to the embodiments illustrated in the drawings, and specific language will be used to describe them. It is to be understood that the Figures are, in some cases, schematic and do not show the various components in their actual scale. In many instances, the Figures show scaled up components to assist the reader.

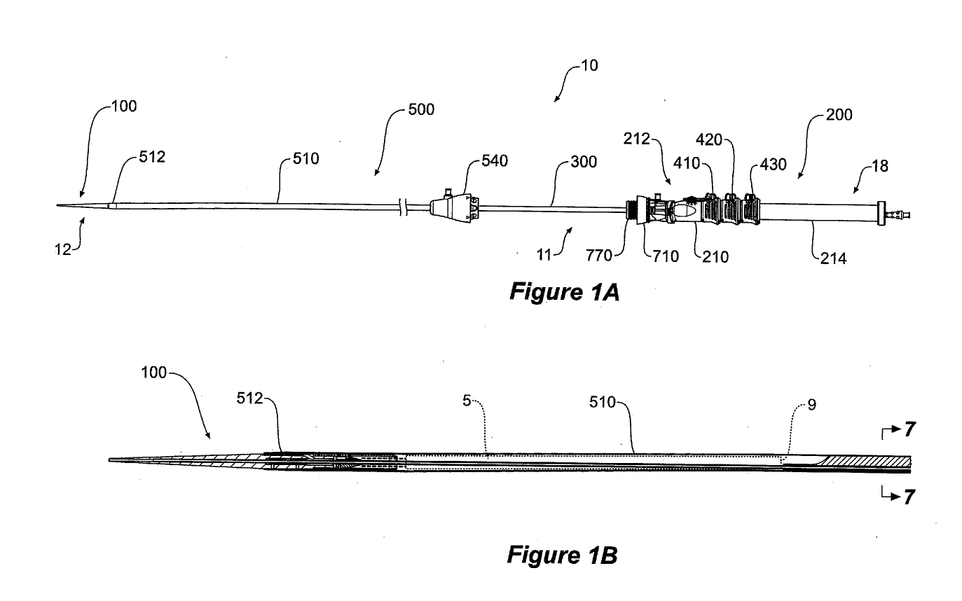

Referring now to FIGS. 1A, 1B, 2A, 2B, 3A, 3B, 4A, 4B, 5A, 5B, 6 and 7, there is shown an endovascular delivery device assembly 10 for the percutaneous insertion into the artery (or other bodily lumen) of prostheses such as stents, stent grafts, catheters, cardiac leads, balloons, and the like, according to a first embodiment of the invention.

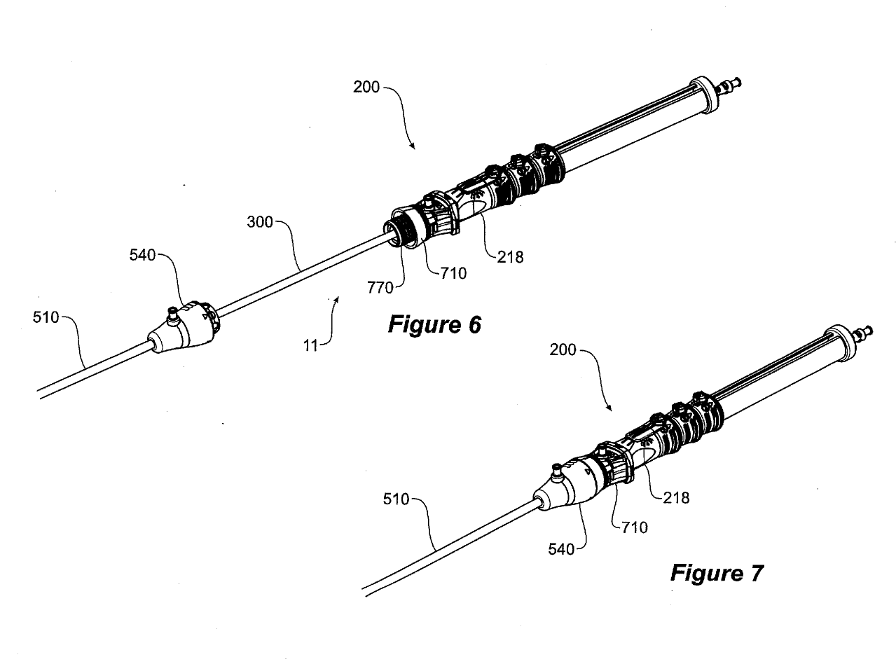

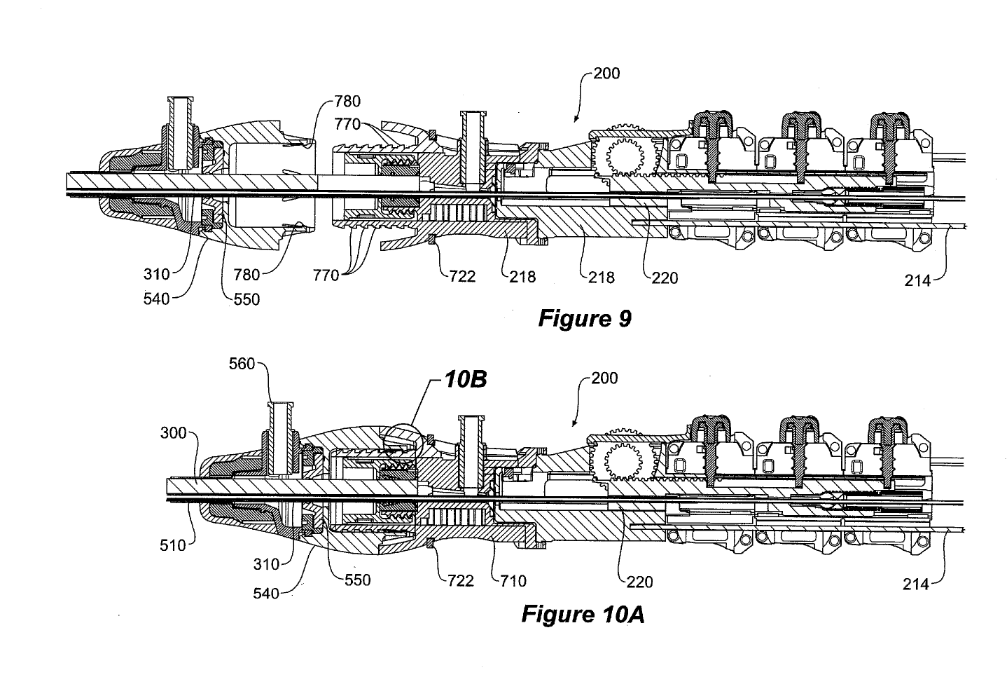

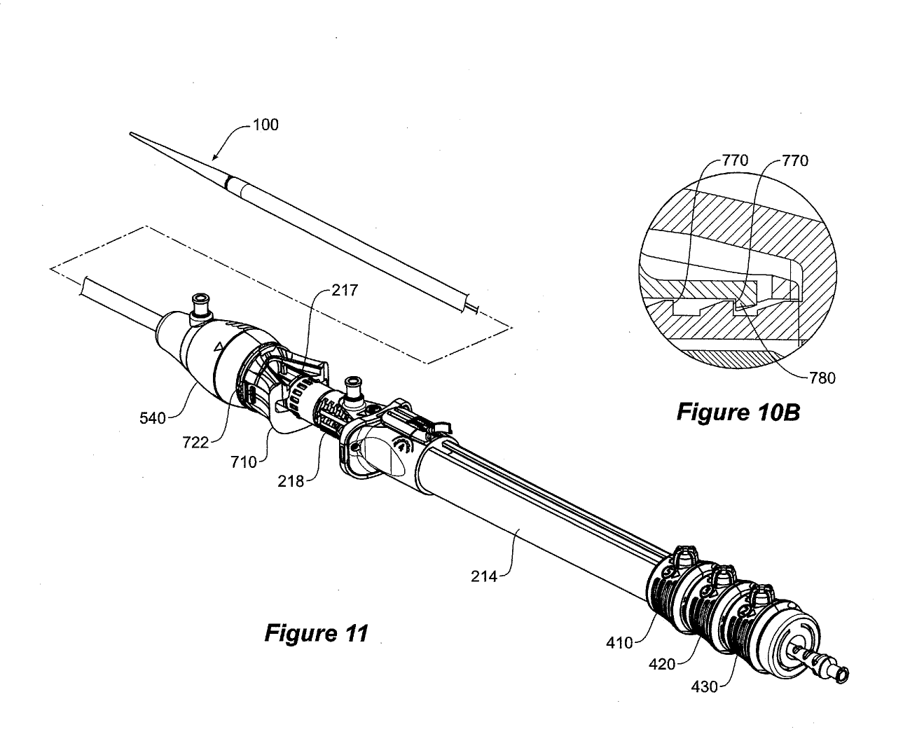

Referring again to FIG. 1A, in conjunction with FIGS. 6 and 7, it can be seen that the endovascular delivery device assembly 10 comprises: a sheath assembly or introducer 500 and a delivery device 11. The introducer 500 includes a seal housing assembly 540 and an elongate sheath 510 extending proximally from the seal housing assembly. The seal housing assembly houses a pusher catheter seal 550 as shown in the cross-sectional views of FIGS. 9 and 10A.

It can be seen from FIG. 1B that the delivery device 11 is loaded with an endograft or stent graft 5.

Referring to FIGS. 1A and 1B, it can be seen that the device has a proximal end 12 and a distal end 18. The delivery device 11 has an outer handle portion 210 mounted to its distal end 18. The outer handle portion 210 has a handle body 218 and a track 214 extending distally from the handle body 218. The handle body 218 is more clearly shown in the cross-sectional view of FIG. 9.

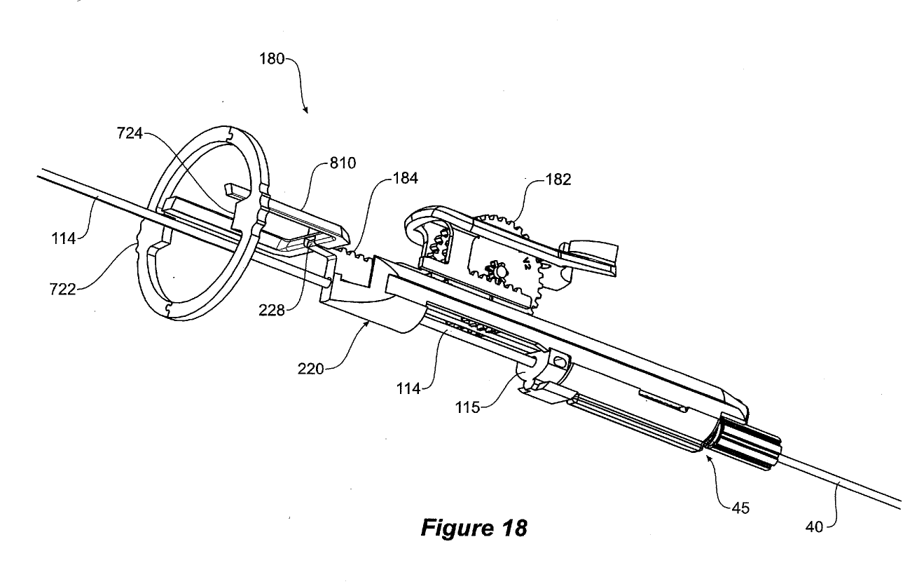

The delivery device 11 also has an inner handle portion 220 which is shown in FIGS. 9 and FIG. 18. The inner handle portion 220 is slidably moveable within, and with respect to, the outer handle portion 210 from a distal position to a proximal position.

A tip assembly 100 is located at the proximal end 12 of the delivery device 11, as is shown in FIGS. 1A and 1B.

The delivery device 11 also has an elongate pusher catheter 300 extending proximally from the handle body 218 towards the tip assembly 100, as can be seen in FIGS. 1B, 2B, 3B and 4B. A distal end of the pusher catheter is attached to the handle body 218. The pusher catheter has a pusher catheter external surface 310, and the pusher catheter extends through the introducer such that the pusher catheter external surface slideably engages with the pusher catheter seal 550 as can be seen in FIG. 9.

A guide wire catheter 40 extends through the inner handle portion 220 and through the pusher catheter 300. The guide wire catheter 40 is affixed at a distal end thereof to the inner handle portion 220 and is affixed at a proximal end 12 thereof to the tip assembly 100. A pin vice assembly 45, the position of which is shown in FIG. 18, can be used. Such a pin vice assembly attaches to the inner handle portion 220, as is shown in FIG. 18. Various pin vice constructions may be used, such as is described in U.S. Pat. No. 7,435,253 to Hartley referred to above, where the pin vice has a screw cap which, when screwed in, clamps vice jaws against the thin walled metal tube (guide wire catheter).

In other embodiments, not shown, gluing, crimping or other attachment means may be used to secure the guide wire catheter 40 to the inner handle portion 220.

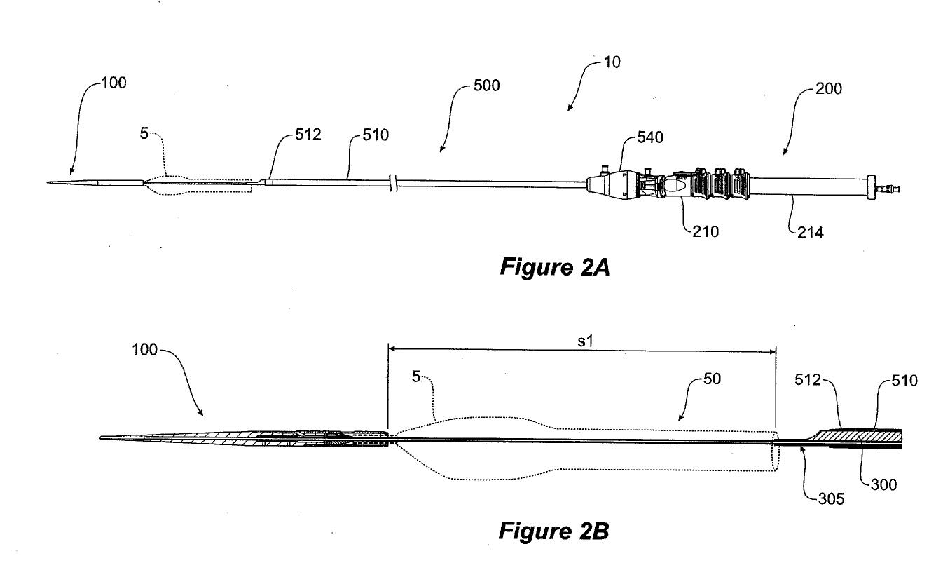

An endograft receiving portion 50, for receiving the endograft 5 between the tip assembly and the pusher catheter, can be seen in FIG. 2B. It should be understood that ‘between’ in this context, means that at least a portion of the endograft receiving portion 50 is between the tip assembly 100 and the pusher catheter 300.

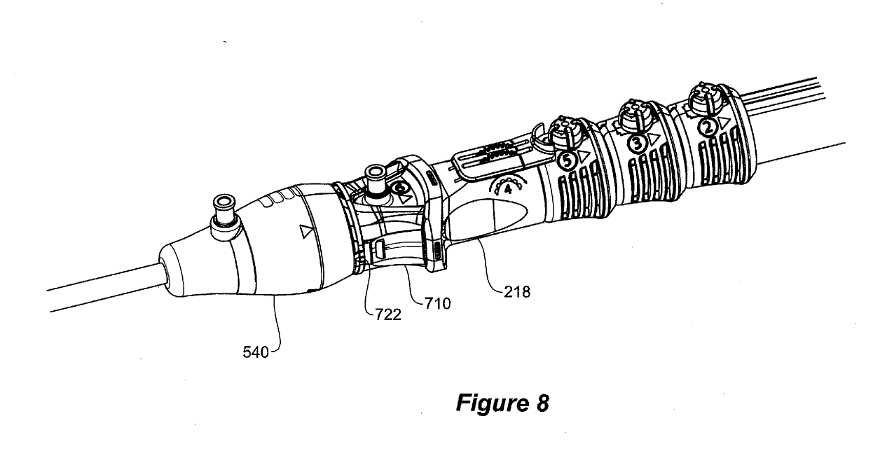

Referring to FIGS. 6, 7 and 8, it can be seen that the assembly further comprises a linking portion 710 for linking the handle body and the seal housing assembly 540 together. The linking portion 710 is mounted between the handle body 218 and the seal housing assembly 540. Referring now to FIGS. 9 and 10, it can be seen that the linking portion 710 has an unlinked condition, shown in FIGS. 6 and 9, and a linked condition, shown in FIGS. 7, 8, 10A and 10B. In the linked condition, sliding movement between the pusher catheter 300 and the pusher catheter seal 550 is limited such that disengagement between the pusher catheter external surface 310 and the pusher catheter seal 550 is prevented.

FIGS. 10A and 10B shows that in the linked condition, the linking portion 710 is joined to the seal housing assembly 540.

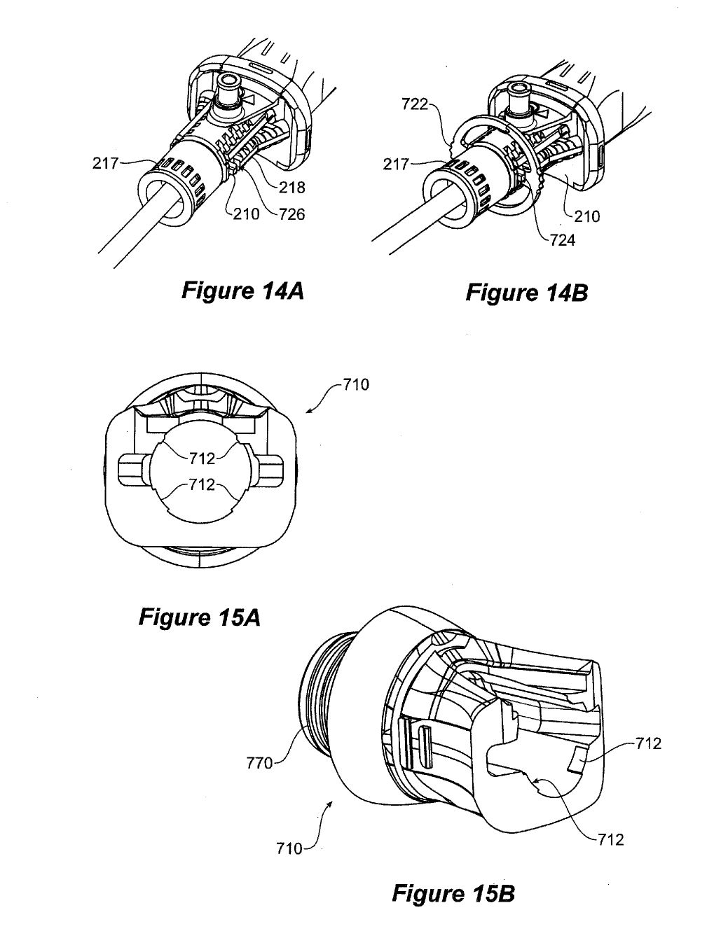

The linking portion 710 is shown as an isolated component in FIGS. 15A and 15B. In these Figures, it can be seen that the linking portion 710 includes four extension limiting projections in the form of tabs 712. These tabs 712 are arranged to engage with a corresponding flange 217 located at a proximal end of the handle body 218 of the outer handle portion 210. The flange 217 is most clearly shown in FIGS. 14A and 14B. FIGS. 11, 12 and 13, particularly FIG. 13, shows how the flange 217 and the tabs 712 engage to limit the sliding movement between the pusher catheter 300 and the pusher catheter seal 550, once the linking portion 710 has been joined to the seal housing assembly 540 as is described below and is shown, for instance, in FIGS. 10A and 10B.

The assembly 10 comprises a joiner assembly for joining the linking portion 710 to the seal housing assembly 540. In the embodiment of the invention illustrated in the figures described above, including FIGS. 6 to 10, the joiner assembly comprises a first mating joiner feature 770 on the linking portion 710. The first mating joining feature 770 is shown in FIG. 1A, FIG. 6, the cross-sectional view of FIG. 13 and in the isometric view Figure of 15B. The joiner assembly further comprises a second mating joining feature 780, which is most easily seen in FIG. 9.

Now referring to FIGS. 9 and 10A, it can be seen that in FIG. 9 the mating feature 780 is longitudinally spaced apart from the first mating feature 770. In FIGS. 10A and 10B, the mating features 780 and 770 are in engagement.

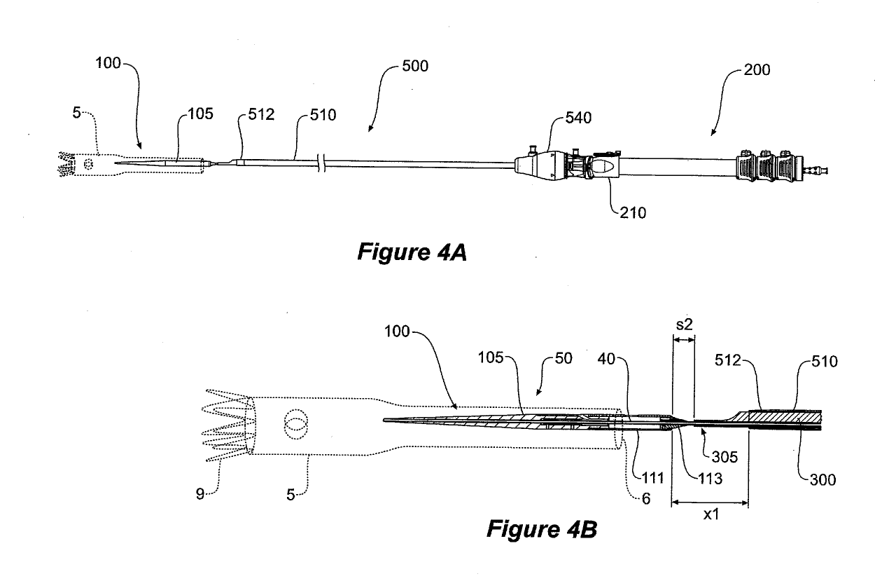

Referring again to FIGS. 2A and 2B, it can be seen that the pusher catheter 300 has a proximal end 305. It can also be seen that the assembly 10 has a first condition, as shown in FIGS. 2A and 2B, in which the proximal end 305 of the pusher catheter 300 is spaced from the tip assembly 100 at a first spacing s1 for accommodating a compressed endograft 5. The assembly further has a second condition in which the proximal end 305 of the pusher catheter 300 is spaced from the tip assembly 100 at a second spacing s2, as is illustrated in FIG. 4B. The second spacing s2 is shorter than the first spacing s1. Furthermore, the second spacing s2 may be substantially zero. That is, the second spacing s2 may be an engagement, depending on the configuration of the tip assembly 100.

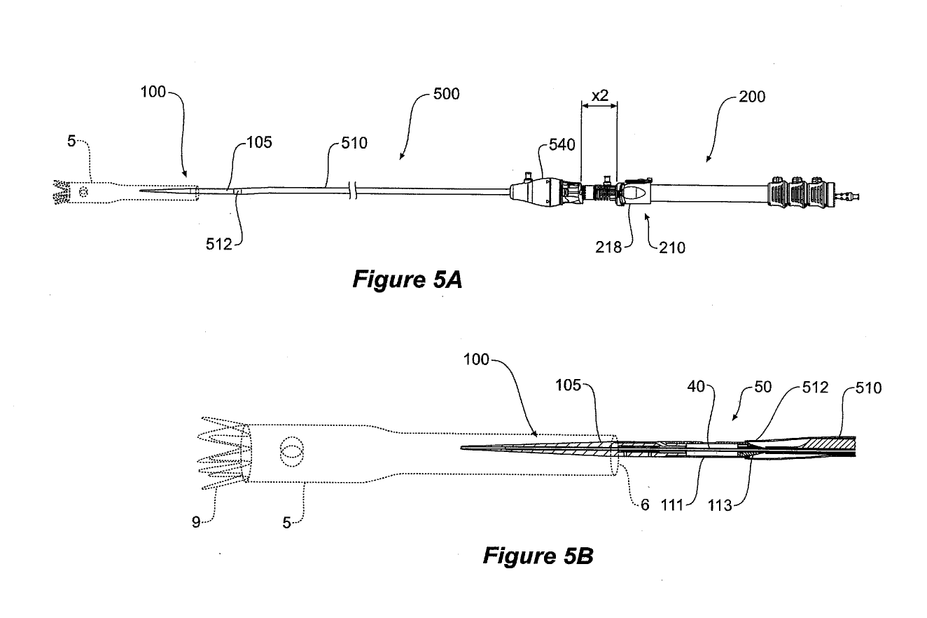

The sheath 510 includes a proximal end 512 shown in FIG. 4B. The assembly 10 has a third condition in which the proximal end 512 of the sheath 510 is adjacent to the tip assembly 100, as is shown in FIG. 5B.

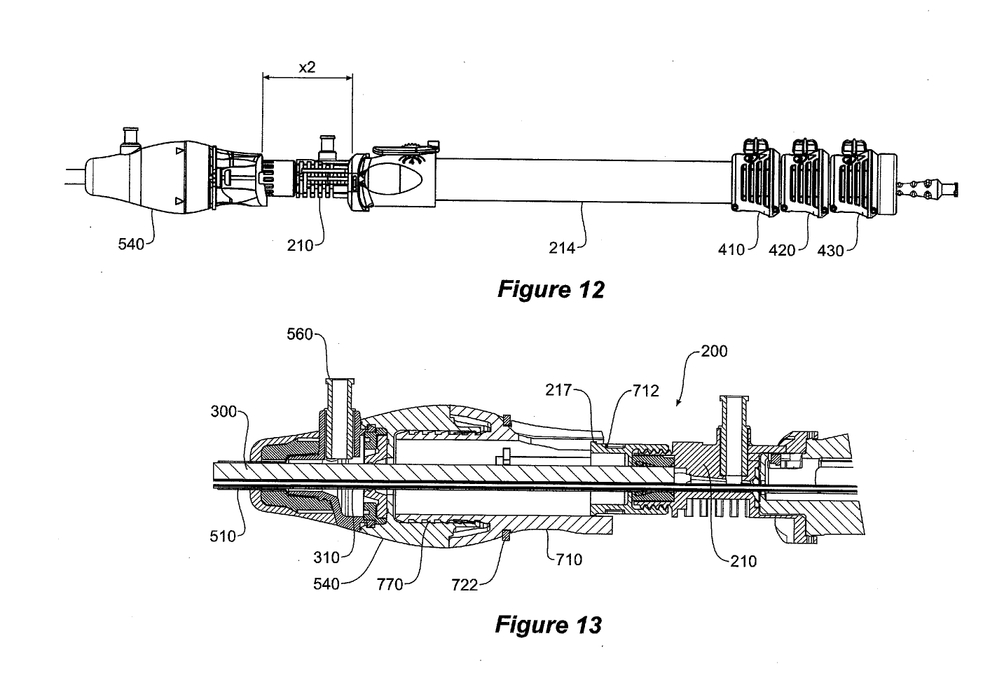

In the linked condition, the limited sliding movement between the pusher catheter 300 and the pusher catheter seal 550 is sufficient to bring the proximal end 512 of the sheath 510 into engagement with the tip assembly 100, as is shown in FIGS. 5A and 5B. This distance is indicated by the length x1 shown in FIGS. 5A and 12.

Referring again to FIGS. 9, 10A, 12 and 13, a lock comprising a locking actuator 722 is shown. The locking actuator 722 has a locking projection 724 which engages with a locking recess 726. The locking recess is shown in FIG. 14A. In FIG. 14B, the locking projection 724 can be seen in its engaged position within the locking recess 726. Reading FIGS. 9, 10, 14A and 14B together, it can be seen that the lock comprises the locking actuator 722 and the locking recess 726. In the locked condition, shown in FIGS. 9, 10A and 14B, the locking projection 724 engages the locking recess 726 to the handle body 218 so as to prevent relative sliding movement. That is, to prevent relative sliding movement between the linking portion 710 and the handle body 218.

FIGS. 17A and 17B also show the locking actuator 722 and its mounting position.

The actuator 722 is movable to an unlocked condition in which the locking projection 724 is disengaged from the locking recess 726. This is achieved through rotation of the locking actuator 722. In the unlocked condition, shown in FIGS. 12 and 13, the linking portion 710 and the handle body 218 are unlocked so as to allow relative sliding movement to the position shown in FIGS. 12 and 13 from the position shown in FIGS. 10A and 10B.

It is important to note that in a preferred embodiment of the invention, illustrated and described herein, the length of the above-mentioned sliding movement is arranged to match the distance required to move from the position shown in FIG. 4B to the position shown in FIG. 5B. That is, the distance x2 shown in FIG. 12 is the same, or about the same, as x1 shown in FIG. 4B. Preferably, x2 is just a little longer than x1 so that the sheath 510 and the tip assembly 100 marry up, as is shown in FIG. 5B with slight overlap. The configuration shown in FIG. 5B allows the whole assembly 10 to be withdrawn from the patient with an atraumatic profile, thereby reducing risks of engagement or catching of either deployed endograft(s), plaque or surfaces within the anatomy of the patient.

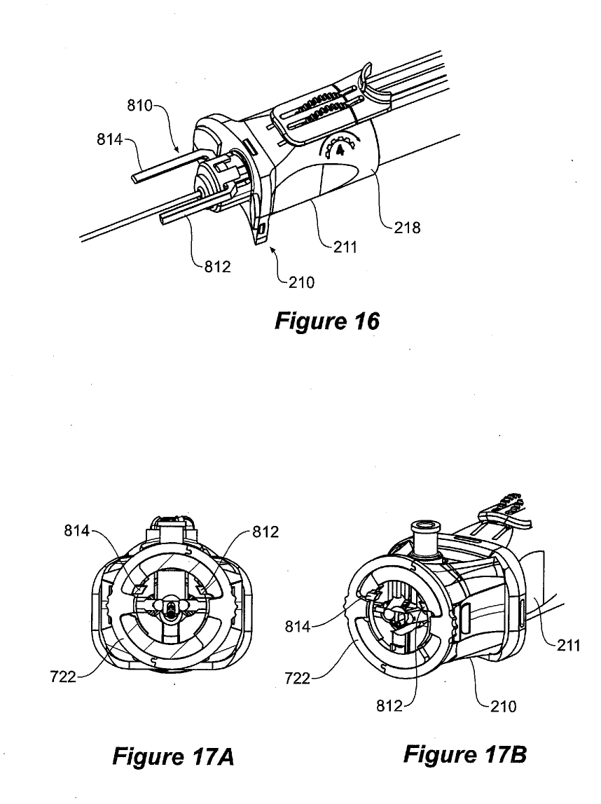

Referring to FIG. 16, which is a detailed isometric view of a component of the handle body shown in FIGS. 14A and 14B, it can be seen that the handle body 218 has a grip portion 211 (also shown in FIG. 17B).

FIG. 16 also shows an optional interlock body 810. The interlock body 810 has legs 812 and 814 that extend longitudinally to a position adjacent to the locking projections 724 of the locking actuator 722 as can be seen in FIGS. 17A, 17B and 18. The interlock body 810 prevents premature actuation of the locking actuator 722. The locking actuator 722 can only be rotated (actuated) after the interlock body 810 has been pulled in a distal direction by the hook 228 shown in FIG. 18. This pulling action only occurs when tip has been retrieved through actuation of the tip retriever. In the embodiment shown in FIG. 18, the tip is retrieved by rotation of actuator 182 (other actuation devices including those employing levers, racks and pinions may also be used).

### Second Embodiment

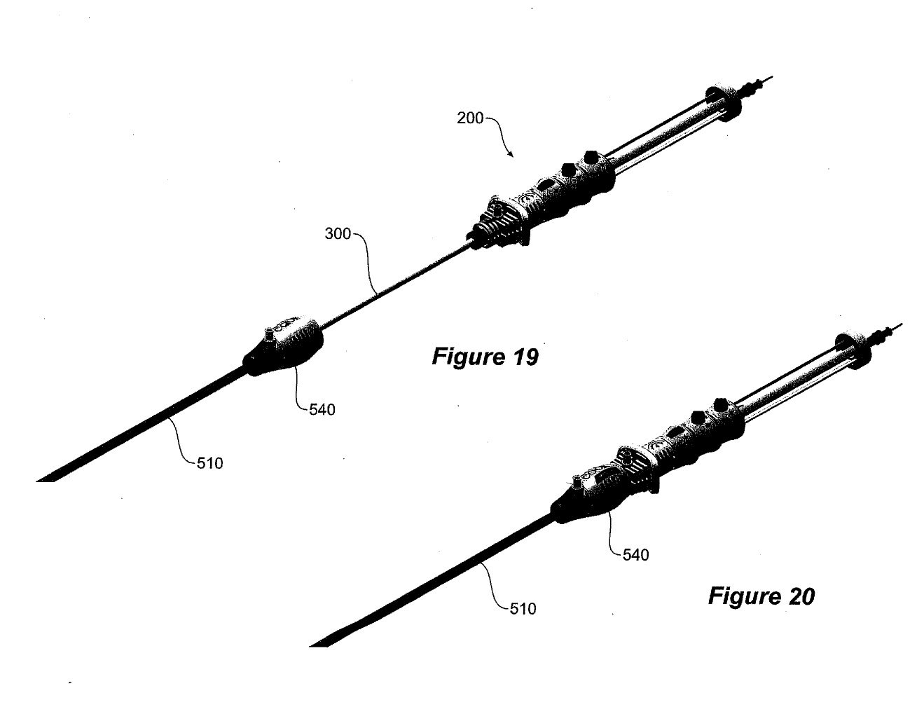

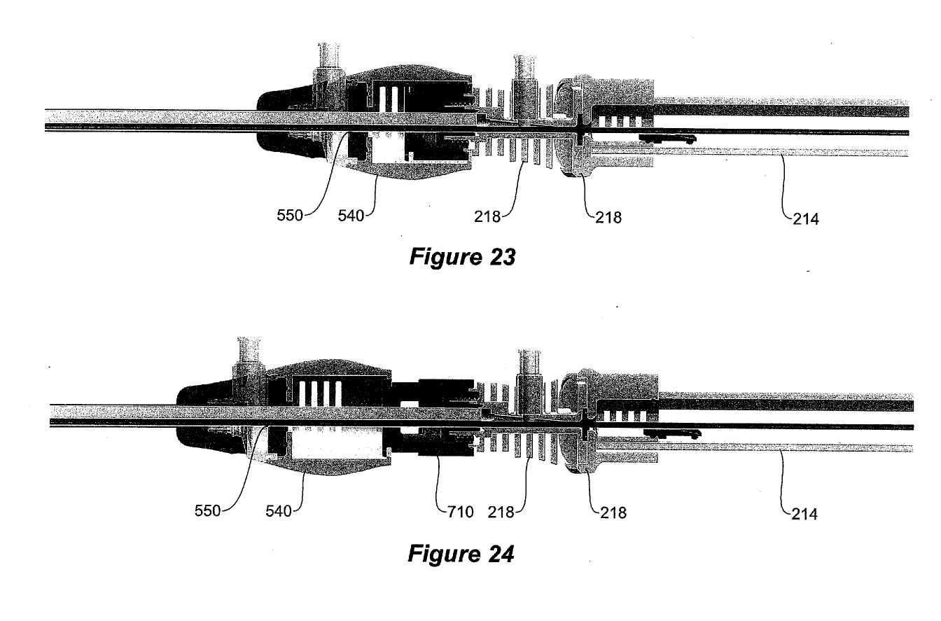

A second embodiment of the invention will now be described with reference to FIGS. 19 to 24.

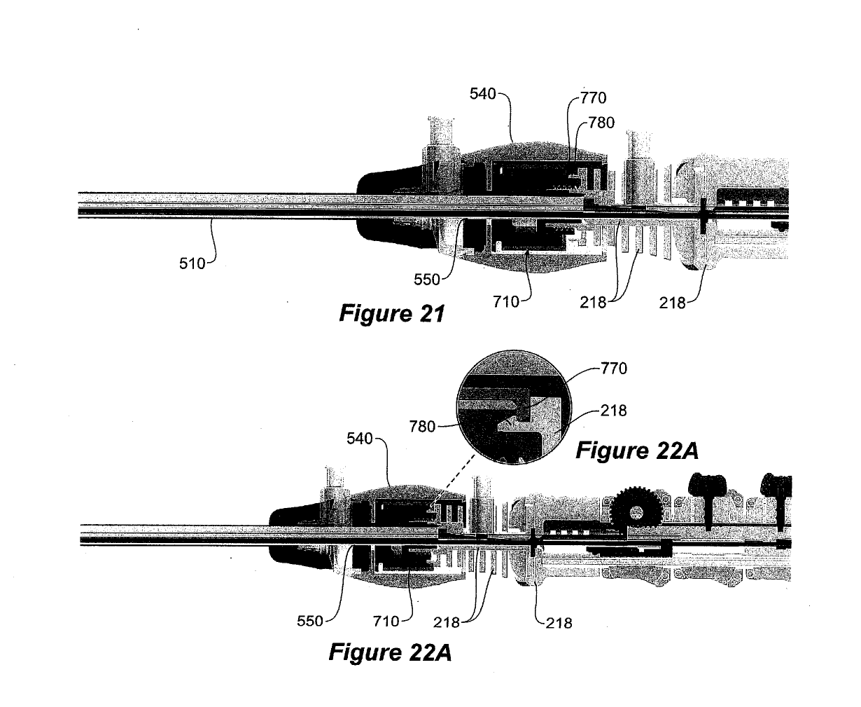

The second embodiment of the invention is of a generally similar construction to the first embodiment of the invention but differs particularly in the area of the linking portion. With the first embodiment of the invention, the linking portion 710 is shown as a separate component in FIGS. 15A and 15B. The linking portion 710 of the first embodiment of the invention is generally external in that it sits around or external to the handle body 218. In contrast, the linking portion 710 of the second embodiment of the invention, which is most clearly shown in the magnified view of FIG. 21, is internal in the sense that it sits more inside the valve body 540. Functionally, the linking portion 710 is similar.

Referring to FIG. 21, it can be seen that the linking portion 710 of the second embodiment of the invention is slidably mounted to the seal housing assembly 540. In FIG. 21, the linking portion is not yet in its linked condition. In transitioning from the position illustrated in FIG. 21 to the position shown in FIGS. 22A and 22B, linking occurs. That is, the linking portion 710 links to the handle body 218. More specifically, linking occurs through the use of a joiner assembly adjoining the linking portion to the handle body. The joiner assembly comprises the first mating feature 770, most clearly shown in FIG. 22B and the linking portion and a second making joiner feature 780 on the handle body 218.

A further feature of the second embodiment of the invention is that the linking portion 710 is in two pieces providing a telescopic action.

### Operation of the Device

Use or operation of the delivery device assembly 10 will now be described. The operation will be described with reference to the first embodiment of the invention.

Referring first to FIG. 1A and its companion FIG. 1B, the delivery device 11 is shown together with a sheath assembly 500 in a configuration ready for use. The combination of the delivery device 11 and the sheath assembly, or introducer, is the delivery device assembly 10.

Typically, one of the first major steps in a procedure undertaken by a vascular surgeon would be to introduce a guide wire into a blood vessel, such as the femoral artery, using the Seldinger technique. This technique involves creating a surgical opening in the vessel of the needle and inserting a wire guide into the vessel through a bore of the needle. The needle is then withdrawn leaving the guide wire in place. The delivery device assembly 10, as shown in FIGS. 1A and 1B, is then inserted over the guide wire and into the vessel.

Once the surgeon has positioned the proximal end 12 of the delivery device assembly 10 near the target delivery area for the endograft 5, the sheath assembly 500 can be withdrawn to the position shown in FIGS. 2A and 2B. In this position, the sheath 510 of the sheath assembly 500 has been pulled back over the compressed endograft for stent graft 5 so as to expose it, as is shown in FIG. 2B. This step is conducted by “grounding” the handle body 218 while pulling the valve body 540 of the sheath assembly 500 in a distal direction (away from the patient). Importantly, this step also engages the linking portion 710 with the seal housing assembly 540 as there is a transition from the position shown in FIG. 9 to the position shown in FIGS. 10A and 10B.

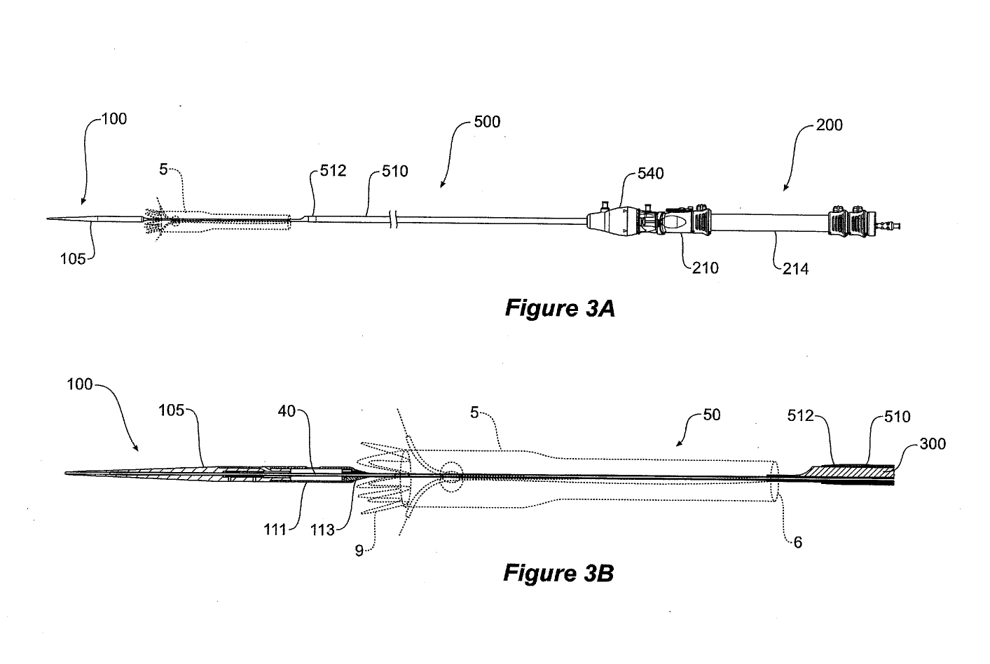

Typically, a next step in operating the delivery device 10 would be causing the stent graft to expand from its reduced condition to an expanded condition. This next step causes removal of a reducing trigger wire. This moves an end of the reducing trigger wire free from the endograft 5 allowing it to expand. The expanded condition can be seen in FIGS. 3A and 3B.

Other steps to deploy the endograft 5 into the patient are generally similar to the steps described in the applicant's earlier PCT patent application number PCT/AU98/00383 titled “A Prosthesis and a Method and Means of Deploying a Prosthesis”.

After this step, the tip assembly 100 is retracted towards the sheath assembly 500 such that the sheath 510 ends up in a position against or over the tip retriever 113, as shown in FIG. 5B, so that the entire delivery device 10 can safely be withdrawn through and out from within the deployed stent graft 5. This retraction step is illustrated in the transition from FIG. 10A to FIG. 11. Retraction is limited by the extent to which the linking portion 710 can slide with respect to the handle body 218. More specifically, the abutment between the flange 217 on the handle body and the tabs 712 on the linking portion 710, most clearly shown in FIGS. 15A and 15B, limits the extent of retraction. The limitation of the retraction is such that disengagement between the pusher catheter 300 and the pusher catheter seal 550 is prevented. This prevents severe blood loss occurring.

Operation of other embodiments of the invention, including the second embodiment of the invention described above would be largely similar.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Please note that the following claims are provisional claims only, and are provided as examples of possible claims and are not intended to limit the scope of what may be claimed in any future patent applications based on the present application. Integers may be added to, or omitted from, the example claims at a later date so as to further define or re-define the invention.

What is claimed is:

1. An endoluminal delivery device assembly comprising:
an introducer, the introducer including a seal housing assembly and an elongate sheath extending proximally from the seal housing assembly, the seal housing assembly housing a pusher catheter seal;
a delivery device, the delivery device including a handle body and an elongate pusher catheter extending proximally from the handle body, the pusher catheter having a pusher catheter external surface, the pusher catheter extending through the introducer such that the pusher catheter external surface slideably engages with the pusher catheter seal;
a tip assembly at a proximal end of the delivery device;
a guide wire catheter extending through the handle body and through the pusher, the guide wire catheter being affixed at a proximal end thereof to the tip assembly;
an endograft receiving portion for receiving the endograft between the tip assembly and the pusher; and
a linking portion for linking the handle body and the seal housing assembly together, the linking portion mounted between the handle body and the seal housing assembly, the linking portion having an unlinked condition and a linked condition,
whereby, in the linked condition, sliding movement between the pusher catheter and the pusher catheter seal is limited such that disengagement between the pusher catheter external surface and the pusher catheter seal is prevented.

2. The assembly as claimed in claim 1 wherein, the linking portion is slideably mounted to the handle body.

3. The assembly as claimed in claim 2 wherein, in the linked condition, the linking portion is joined to the seal housing assembly.

4. The assembly as claimed in claim 3 comprising a joiner assembly for joining the linking portion to the seal housing assembly.

5. The assembly as claimed in claim 4 wherein the joiner assembly comprises:
a first mating joiner feature on the linking portion; and
a second mating joiner feature on seal housing assembly.

6. The assembly as claimed in claim 5 wherein the pusher catheter includes a proximal end, and wherein the assembly comprises:
an first condition in which the proximal end of the pusher catheter is spaced from the tip assembly at a first spacing for accommodating a compressed endograft; and
a second condition in which the proximal end of the pusher catheter is spaced from the tip assembly at a second spacing, the second spacing shorter than the first spacing.

7. The assembly as claimed in claim 6 wherein the sheath includes a proximal end, and wherein the assembly has a third condition in which the proximal end of the sheath is adjacent to the tip assembly.

8. The assembly as claimed in claim 7 wherein, in the linked condition, the limited sliding movement between the pusher catheter and the pusher catheter seal is sufficient to bring the proximal end of the sheath into engagement with the tip assembly.

9. The assembly as claimed in claim 5 comprising a lock, the lock comprising:
a locking actuator having a locking projection; and
a locking recess,
wherein the lock has a locked condition in which the locking projection engages the locking recess thereby locking the linking portion to the handle body so as to prevent relative sliding movement.

10. The assembly as claimed in claim 9 wherein the actuator is moveable to an unlocked condition in which the locking projection is disengaged from the locking recess.

11. The assembly as claimed in claim 1 wherein, in the unlinked condition, the linking portion is slideable mounted to the seal housing assembly.

12. The assembly as claimed in claim 11 wherein, the linking portion is slideably mounted to the seal housing assembly.

13. The assembly as claimed in claim 12 wherein, in the linked condition, the linking portion is joined to the handle body.

14. The assembly as claimed in claim 3 comprising a joiner assembly for joining the linking portion to the handle body.

15. The assembly as claimed in claim 14 wherein the joiner assembly comprises:
a first mating joiner feature on the linking portion; and
a second mating joiner feature on handle body.
